(19) United States
(12) Patent Application Publication
(10) Pub. No.: US 2009/0192517 A1
(43) Pub. Date: Jul. 30, 2009

(54) DYNAMOMETRIC TOOL FOR MEDICAL USE

(75) Inventors: Philippe MILETTO, Orny (CH); Silver GROSS, La Chaux-deFonds (CH)

Correspondence Address: YOUNG & THOMPSON, 209 Madison Street, Suite 500, ALEXANDRIA, VA, 22314, US

(73) Assignee: HADER SA, La Chaux-de-Fonds (CH)

(21) Appl. No.: 12/361,806

(22) Filed: Jan. 29, 2009

(30) Foreign Application Priority Data
Jan. 29, 2008 (EP) 08101067.0

(51) Int. Cl. A61B 17/58

(52) U.S. Cl. 606/104

(57) ABSTRACT

A dynamometric tool for medical use with a longitudinal axis AA includes: a hollow grip (10) including an interior wall having a succession of recesses (29) oriented along the axis AA and the section of which is egg-shaped, an instrument holder (30) to be secured in rotation with an instrument configured to cooperate with an object to be screwed, the instrument holder being extended by a shaft (18) with an axis AA pivoting inside the grip. The instrument holder (30) is frictionally connected to the grip using a plurality of elastic members (2) arranged between the shaft (18) and the grip (10), each elastic member (2) including a plurality of blades (4) arranged primarily along non-radial directions and elastically deformable along an essentially radial direction, the ends of the blades forming skids designed to cooperate with the recesses (29) of the interior wall.

### TECHNICAL FIELD

The present invention relates to the field of tools for medical use. It more particularly concerns a dynamometric tool designed to tighten and loosen screws or various objects comprising a screw pitch, typically during a surgical operation.

It is, indeed, particularly important to avoid applying uncontrolled tightening torques, for example in the case where a plate is fixed on a bone to repair a fracture. If the tightening applied is excessive, this can lead to crushing the bone and damaging it further.

### PRIOR ART

One knows, in the prior art, dynamometric tools for screwing, and sometimes unscrewing, of various objects, in particular screws for fixing reconstruction elements in plastic surgery. As an example of application, plates can be screwed into fractured bones in order to facilitate their healing. A tool of this type comprises:
a grip so that the surgeon can manipulate it, and
an instrument holder to be secured in rotation with an instrument configured to cooperate with the object to be screwed.

There are two main types of dynamometric tools. A first provides a limitation of the tightening torque, through the establishment of a safety stop which makes the grip and the instrument holder secured in rotation as long as the selected maximum torque is not reached and which stops when a determined tensile strength corresponding to the maximum torque is reached. This type of tool is described, for example, in document U.S. Pat. No. 5,368,480. A torque limiting member is inserted between the grip and the instrument holder. This member comprises a plurality of lugs dimensioned so as to form the abovementioned stop and to collapse when the desired tightening torque is reached.

However, these lugs undergo a plastic deformation, i.e. irreversible, the result of which is that each of the lugs can only be used a single time. Of course, the fact that the torque limiting member comprises a plurality of lugs makes it possible to perform several tightenings at maximum torque, but only a very limited number. Moreover, it is difficult to precisely control the maximum tightening torque, because the plastic deformation threshold depends on a large number of parameters and can be influenced by slight differences having taken place during manufacturing of the torque limiting member.

The second type of dynamometric tools makes it possible to perform a large number of tightenings at the maximum torque, without having to intervene on the tool, thanks to a sort of snapping system. In certain tools of this type, the instrument holder is extended by a shaft pivoting in the grip, the shaft being frictionally connected to the grip. The friction is provided by two Breguet toothings, i.e. saw-shaped serrated toothings, one being integral with the instrument holder and the other being integral in rotation with the grip. Springs press the two Breguet toothings against each other so as to make the instrument holder and the grip integral in rotation. When the tightening torque is greater than the friction imposed between the two Breguet toothings by the springs, the latter parts rub against each other and escape. The instrument holder is then no longer driven in rotation by the grip. The maximum applicable tightening torque can be adjusted by modulating the pressure exerted by the springs.

The friction created between the toothings is particularly significant and it is necessary, in order to obtain acceptable precision and longevity of the tool, for the Breguet toothings to be metal. They, as well as the springs, are made in stainless steel, allowing surgical use which is as hygienic as possible. It is, however, necessary to grease the metallic parts in friction, which is not very satisfactory from a sanitary perspective, since a risk of grease flow outside the tool exists during sterilization operations. Moreover, the precision of such a tool is not very satisfactory (±10%) and it is necessary to perform calibrations regularly.

Furthermore, when the maximal tightening torque is reached and the toothings escape each other, this causes jumps in the longitudinal direction of the tool, which is not pleasant for the surgeon and can cause him to make a clumsy gestures. Moreover, the materials used to produce this tool make it heavy and not very practical.

The present invention concerns dynamometric tools of the second type, making it possible to perform a large number of tightenings at the maximum torque, without intervention on the tool. The aim sought is to propose a dynamometric tool free of the aforementioned drawbacks and which, in particular, is precise, light, easy to manipulate. Furthermore, the invention also aims to propose an improved tool, the maximum tightening torque of which is not affected by the treatments undergone during the sterilization process.

### BRIEF DESCRIPTION OF THE INVENTION

More precisely, the invention concerns a dynamometric tool for medical use, with a longitudinal axis AA comprising:
a hollow grip comprising an interior wall having a succession of recesses oriented along the axis AA and the section of which is egg-shaped,
an instrument holder to be secured in rotation with an instrument configured to cooperate with an object to be screwed, said instrument holder being extended by a shaft with axis AA pivoting inside the grip.

The instrument holder is frictionally connected to the grip using a plurality of elastic members arranged between the shaft and the grip. Each elastic member comprises a plurality of blades arranged primarily along non-radial directions and which are elastically deformable along an essentially radial direction, the ends of the blades forming skids designed to cooperate with the recesses of the interior wall.

According to the invention, in order to avoid the expansion caused by heating from sterilization causing a modification of the elastic properties of the elastic members and an upset of the maximum tightening torque, the recesses succeed each other without interruption, such that, between two consecutive recesses, there is no surface allowing a skid to occupy a stable position.

### BRIEF DESCRIPTION OF THE DRAWINGS

Other details will appear more clearly upon reading the following description, done in reference to the appended drawings, in which:

FIG. 1 is a transverse cross-section of the grip and an elastic member particularly adapted for the implementation of the invention, the figure including a close-up showing the interaction between the grip and an elastic member in detail, and

FIG. 2 is a longitudinal cross-section of the device according to the invention of a zone of this part, and

FIGS. 3 and 4 show, in top view and perspective view, an embodiment of an elastic member which can advantageously be used in the tool according to the invention.

### DETAILED DESCRIPTION OF THE INVENTION

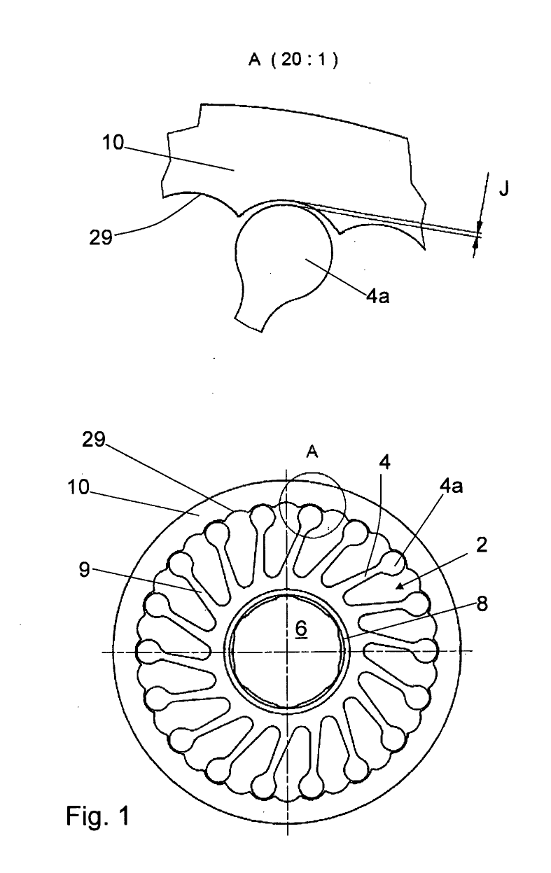

The dynamometric tool according to the invention implements a torque limiting member which is an elastic member 2 illustrated in FIG. 1. The elastic member 2 is generally cylindrical in shape and comprises, in its center, an opening 6 structured so as to have a non-circular section, not elastically deformable, and forming a female member.

More particularly, the elastic member 2 comprises a plurality of blades 4 whereof the shape, given the material used, is determined such that they are elastically deformable along an essentially radial direction. These blades 4 are oriented toward the outside, primarily along non-radial directions and end, in one preferred embodiment, with a skid comprising, for example, a cylindrical portion 4a substantially orthogonal to the general plane of the elastic member. As one will understand below, it is the elastic deformation of the blades which makes it possible to obtain the limiting of the torque. Contrary to a torque limiting member of the prior art which deforms plastically, the elastic member 2 used in the invention does not undergo any irreversible transformation when the maximum torque is reached.

As one can see in FIG. 1, the opening 6 can also be arranged in a hub 8 realized using a second piece, integral with a bushing 9 supporting the blades 4. The hub is advantageously realized in a material which is not elastically deformable, for example of the metal type, such as a stainless steel.

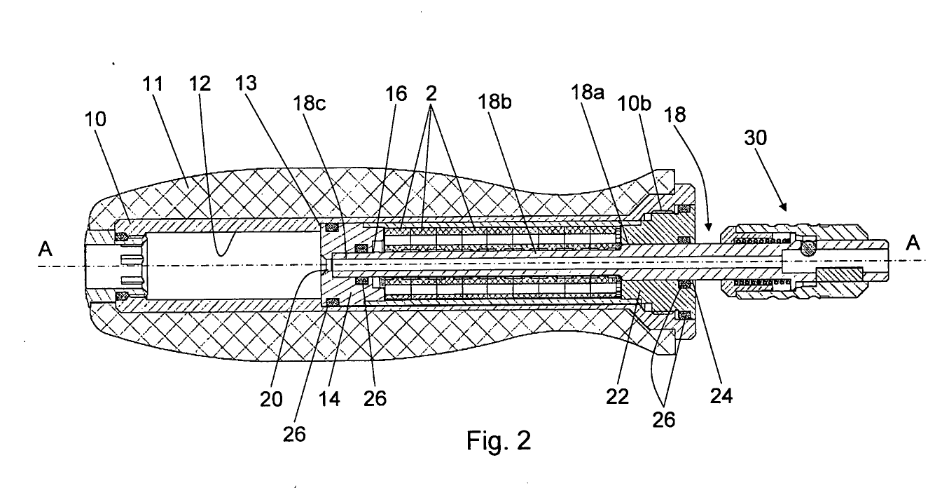

FIG. 2 shows a grip 10, with a longitudinal axis AA, topped with a handle 11. Along this axis, the body 10 is passed through by a cylindrical channel 12. The latter part comprises a stop 13 designed to provide support for a first bearing 14, having, in its center, a housing 16 to receive a shaft 18, which will be described in detail below. The shaft 18 can comprise, at its end, a tapping 20 with axis AA designed to cooperate with a screw in order to secure it to the bearing 14. One can, for example, access the screw from the end of the grip.

The second end of the grip 10 is provided with a screw pitch 10b in order to cooperate with a second bearing 22, provided in its center with an opening 24 in which the shaft is adjusted.

At each of the bearings 14 and 22, joints 26 can be inserted between the bearing and the grip 10, on one hand, and between the bearing and the shaft 18, on the other hand.

We have also illustrated, in FIG. 2, an instrument holder 30 of the type known by one skilled in the art, likely to be secured in rotation to an instrument configured to cooperate with an object to be screwed. The part of the instrument holder providing the connection with the instrument is not in itself part of the invention and will not be described in detail.

The instrument holder 30 is extended by the shaft 18 previously mentioned. This is dimensioned so as to be able to go through the opening 24, assume a position in the channel 12 of the grip 10, while its free end assumes a position in the housing 16. More specifically, the shaft 18 comprises a first portion 18a adjusted to the dimension of the opening 24. Then, when going away from the part of the instrument holder designed to receive an instrument, a second portion 18b designed to be housed in the channel 12. The second portion 18b is structured so as to have a non-circular section, and thereby forms a male member able to be connected in rotation with the female member of the elastic member 2, which has a corresponding shape. Lastly, the shaft ends with a third portion 18c adjusted to the dimensions of the housing 16.

The instrument holder 30 is designed to be mounted pivoting, frictionally, in the channel 12, using a plurality of elastic members 2 mounted on the shaft 18 between the two bearings 14 and 22, interposed between the shaft 18 and the wall of the channel 12.

In order to ensure the friction between the grip 10 and the elastic members 2, the interior wall of the channel 12 has a succession of recesses 29, oriented along the axis AA and whereof the section, visible in FIG. 1 and particularly in the close-up view, is egg-shaped. The recesses 29 occupy the entire length of the channel 12 between the two bearings 14 and 22, and are designed to receive the skids.

The cylindrical portions 4a are thereby defined so as to cooperate with the recesses 29 of the channel 12. Other forms may be chosen as long as the ends of the blades 4 are able to cooperate with the structures of the wall of the channel 12 to create a friction likely to stress the elastic blades 4. More particularly, it is the stress which it is necessary to apply on the elastic blades 4 so that they go from one recess to the other which determines the maximum tightening torque.

It has been noted that tools having the abovementioned characteristics could undergo fluctuations of their maximum tightening torque after they had undergone sterilization treatments. To offset this drawback, one of the important aspects of the invention illustrated in the close-up of FIG. 1, the recesses succeed each other without interruption, such that, between two consecutive recesses 29, there is no surface allowing a skid to occupy a stable position. In other words, the interior wall of the channel 12 is configured such that only the recesses 29 can receive the skids stably. The zone which can be defined between two recesses is arranged so as not to allow the skids to rest there stably. Indeed, if this characteristic is not respected, the elastic members 2 can be positioned stably with the skids resting between two recesses 29. Naturally, in such a position, the elastic blades 4 are stressed. If sterilization is done while the elastic members are thus stressed, their expansion caused by heating from the sterilization causes a modification of the elastic properties of the members 2 and an upset of the maximum tightening torque.

Furthermore, the elastic members 2 are mounted on the shaft 18, integral in rotation but with play. First of all, one will ensure that play remains between the male member formed by the shaft 18 and the female member formed by the opening 6. This play is typically between 1 and 5 hundredths, preferably 3 hundredths. Then, a clearance J between 2 and 15 hundredths of millimeters, preferably between 5 and 10 hundredths is provided between the skids and the recesses 29, when the elastic members are at rest. One will note that the elastic members 2 will not be idle on the shaft if they only have one play between the male and female members. Indeed, in such a case, the blades 4 would press on the interior wall of the channel 12, which would maintain the elastic members. The combination of these plays makes it possible to avoid any hyperstaticity phenomenon in the elastic members 2. Thus, when, during the sterilization steps, the elastic members 2 expand under the effect of the heat, this deformation takes place freely, without creating excessive stress on the elastic members which, after cooling, keep all of their elastic and nominal properties. The maximum tightening torque is thus perfectly conserved.

In order to obtain perfect alignment of the elastic members 2 in reference to the axis AA and the grip 10, despite the aforementioned plays, the role and precision of the bearings 14 and 22 are important. Indeed, only the bearings serve as guide element for the shaft 18. Moreover, by its screwing, the second bearing 22 positions the elastic members 2 along the axis AA.

The elastic members 2 are advantageously realized in a material not requiring lubrication and resisting the usual sterilization treatments, thermal and by radiation. Various tests have made it possible to demonstrate that polymers of the polyether-ether-ketone type (known under the name PEEK) had the necessary characteristics. One could more precisely choose PEEK 151G. One could also realize the grip 10 in PEEK, or coat the interior wall of the channel 12 with it, the elastic members being realized in another material able to have suitable elastic properties, in stainless steel, for instance. One skilled in the art could also consider realizing the elastic members, on one hand, and the grip on the other, in a single material not requiring lubrication. One such possibility is particularly advantageous insofar as it eliminates all difficulties related to differences in the expansion coefficient between the elastic members 2 and the grip 10.

Typically, elastic members as described above and made in PEEK make it possible to obtain tightening torque values in the vicinity of several N.m, typically between 0.5 and 15 N.m.

Thus is obtained a tool whereof the adjustment of the maximum tightening torque can be particularly precise, with an improved lifespan. Not only does the tool have the advantages of certain tools of the prior art, namely that the precision achieved is in the vicinity of 3% for 10,000 releases. A release is defined as being the moment when the grip separates from the instrument holder, going from a first to a second relative position of one of these elements in relation to the other.

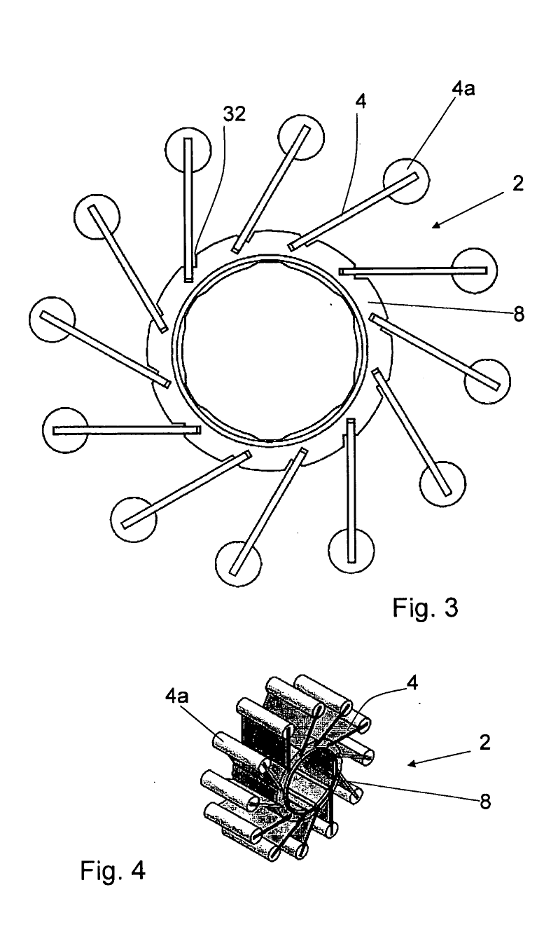

FIGS. 3 and 4 show one particular embodiment of an elastic member 2 which can be used in a tool according to the invention. An elastic member of this type comprises a hub 8 which is cylindrical in shape, the inner wall of which is dimensioned and configured similarly to what was previously described. The outer wall comprises, regularly distributed on its entire perimeter, grooves 32 arranged along the longitudinal axis of the hub and oriented, in the direction of their depth, along a non-radial direction. More particularly, the grooves 32 all have an identical orientation relative to the hub 8. Preferably, each groove occupies the entire height of the hub.

In this embodiment, the elastic blades 4 are each made up of a steel tab, having a rectangular shape, a first end of which is housed in each groove. The grooves 32 are dimensioned such that the tabs are housed without play in the grooves. The tabs are fixed, for example by welding, in the grooves. At their other end, the blades receive a cylinder 4a forming a skid, the cylinder being molded from a casting, for example, and produced in PEEK or in a similar material not requiring lubrication and resisting the usual sterilization treatments, thermal and by radiation.

An elastic member of this type has a particularly interesting reactivity, which is to say that the blades react particularly quickly going from one recess to the next, which allows excellent control of the frictional torque, even in case of quick actuation of the tool. Furthermore, a member of this type allows the application of a more significant force, increased by approximately 50% relative to a member with PEEK blades of the same dimensions.

One will also note that, with a grip whereof the inner wall is provided with thirty-six recesses 29, it is possible to use elastic members provided with 12 or 18 elastic blades, which makes it possible, with a single tool, to obtain torques of k or with a value of 1.5 k, simply by changing elastic members. It has been possible through tests to verify that elastic members of this type keep a constant torque for 50,000 releases.

Thus, the tool according to the invention does not need to be lubricated and can be sealed, avoiding all risk of contamination of the patient. Moreover, it has the essential advantage of behaving particularly well during heating related to sterilization operations. Even after repeated heating cycles, the maximum torque calibrated in the factory remains unchanged, this guarantees extremely safe and secure use.

1. A dynamometric tool for medical use with a longitudinal axis AA comprising:
a hollow grip including an interior wall having a succession of recesses oriented along the axis AA and the section of which is egg-shaped,
an instrument holder to be secured in rotation with an instrument configured to cooperate with an object to be screwed, said instrument holder being extended by a shaft with an axis AA pivoting inside the grip,
said instrument holder being frictionally connected to said grip using a plurality of elastic members arranged between said shaft and said grip,
each elastic member comprising a plurality of blades arranged primarily along non-radial directions and elastically deformable along an essentially radial direction, the ends of the blades forming skids designed to cooperate with said recesses of said interior wall,
wherein the recesses succeed each other without interruption, such that, between two consecutive recesses, there is no surface allowing a skid to occupy a stable position.

2. The tool of claim 1, wherein said skids are cylindrical in shape and are designed to be substantially parallel to said shaft.

3. The tool of claim 1, wherein said elastic members are mounted idle on the shaft and have play between the shaft and said elastic members, on one hand, and between said elastic members and said interior wall, on the other hand.

4. The tool of claim 2, wherein said elastic members are mounted idle on the shaft and have play between the shaft and said elastic members, on one hand, and between said elastic members and said interior wall, on the other hand.

5. The tool of claim 3, wherein the play between said skids and said recesses is between 2 and 15 hundredths of millimeters, preferably between 5 and 10 hundredths of millimeters, when the tool is at rest, and in that the play between said shaft and said elastic members is typically between 1 and 5 hundredths of millimeters, preferably 3 hundredths of millimeters, when the tool is at rest.

6. The tool of claim 4, wherein the play between said skids and said recesses is between 2 and 15 hundredths of millimeters, preferably between 5 and 10 hundredths of millimeters, when the tool is at rest, and in that the play between said shaft and said elastic members is typically between 1 and 5 hundredths of millimeters, preferably 3 hundredths of millimeters, when the tool is at rest.

7. The tool of claim 1, wherein said shaft is mounted pivoting in the grip using at least first and second bearings, adjusted in the grip, the shaft being adjusted in said bearings, said elastic members being mounted on the shaft, between said bearings.

8. The tool of claim 2, wherein said shaft is mounted pivoting in the grip using at least first and second bearings, adjusted in the grip, the shaft being adjusted in said bearings, said elastic members being mounted on the shaft, between said bearings.

9. The tool of claim 3, wherein said shaft is mounted pivoting in the grip using at least first and second bearings, adjusted in the grip, the shaft being adjusted in said bearings, said elastic members being mounted on the shaft, between said bearings.

10. The tool of claim 5, wherein said shaft is mounted pivoting in the grip using at least first and second bearings, adjusted in the grip, the shaft being adjusted in said bearings, said elastic members being mounted on the shaft, between said bearings.

11. The tool of claim 7, wherein the first bearing is mounted abutting inside said channel, the second bearing being screwed onto the grip.

12. The tool according to claim 1, wherein said elastic members comprise a hub realized in a material which is not elastically deformable and a bushing supporting the blades and mounted secured around the hub.

13. The tool according to claim 1, wherein the elastic blades are realized in PEEK.

14. The tool of claim 12, wherein the elastic blades are realized in PEEK.

15. The tool according to claim 1, wherein the interior wall of said channel is in PEEK.

16. The tool of claim 12, wherein the interior wall of said channel is in PEEK.

17. The tool according to claim 1, wherein the grip is in PEEK.

18. The tool of claim 12, wherein the grip is in PEEK.

19. The tool according to claim 12, in which the elastic blades are each made up of a steel tab, secured by a first of their ends in a groove arranged in the hub, along a longitudinal axis thereof, and comprises, at their second end, said skid.
